(19) United States
(12) Patent Application Publication
(10) Pub. No.: US 2012/0276574 A1
(43) Pub. Date: Nov. 1, 2012

(54) MICROFLUIDIC MIXER

(75) Inventors: Nicol Low, Loanhead Lothian (GB); David Thomson, Hamilton (AU); Jonathan Salmon, Makati (PH)

(73) Assignee: ITI Scotland Limited, Glasgow (GB)

(21) Appl. No.: 13/501,737

(22) PCT Filed: Oct. 11, 2010
(86) PCT No.: PCT/EP10/65228

§ 371 Date: Jul. 20, 2012

(30) Foreign Application Priority Data
Oct. 13, 2009 (GB) 0917917.7

(51) Int. Cl. B01L 3/00; G01N 33/68; B23P 11/00; G01N 1/28; G01N 21/00; C12Q 1/02; G01N 33/50

(52) U.S. Cl. 435/29; 436/86; 436/94; 436/174; 436/164; 422/502; 137/15.01

(57) ABSTRACT

Provided is a microfluidic or nanofluidic apparatus which comprises:

(a) a reservoir for one or more fluids;

(b) a mixing device configured to mix the one or more fluids within the reservoir; and

(c) a microfluidic or nanofluidic device in fluid connection with the reservoir;

wherein the reservoir is suitable for containing a total volume of fluid of from 100 microliters to 100 milliliters. Also provided is a method of manufacturing the apparatus and the use of the apparatus to detect an analyte in a fluid.

### FIELD OF THE INVENTION

The present invention relates to an apparatus for mixing one or more fluids in a microfluidic or nanofluidic device and a method of manufacturing the apparatus.

### BACKGROUND OF THE INVENTION

Microfluidic and nanofluidic devices are well known in the art, and are designed to manipulate fluids that are constrained in the microscale or nanoscale respectively. Microfluidic and nanofluidic devices have been used in many different fields which require the use of very small volumes of fluids, including engineering and biotechnology. For example, microfluidic systems have been used in the development of inkjet printheads and DNA chips.

Microfluidic or nanofluidic devices typically comprise one or more channels for passage of a fluid. In many applications it is useful to provide a homogeneous solution for analysis in the microfluidic or nanofluidic device. This may involve mixing a single solution, for example to agitate a suspension, or mixing two or more different solutions.

It is known to integrate a micromixer with a nanofluidic or microfluidic device, but known micromixers can only handle volumes of fluid in the range of nanoliters to a few microliters. Micromixers are also difficult to manufacture given their small scale.

There is a need to provide larger scale mixers with associated volumes of hundreds of microliters to militers which are straightforward to manufacture and can be easily integrated with nanofluidic or microfluidic devices.

For many applications it is desirable to be able to handle macro scale fluids using a microfluidic platform, especially in a diagnostic environment where target molecules might need to be detected at ultra low concentrations (for example 10 virus particles per millilitre of blood for the diagnosis of Hepatitis C virus). In the case of ultra low concentrations, larger volumes have to be used in order to provide a sufficient number of molecules for detection. Thus it is desirable to provide a microfluidic or nanofluidic device which integrates larger volumes of fluids and enables sufficient mixing of the fluids whilst still taking advantage of the microfluidic approach.

Accordingly, it is an aim of the present invention to solve one or more of the problems with the prior art described above. Specifically, it is an aim of the present invention to provide a microfluidic or nanofluidic device which comprises a mixing device which can handle larger volumes of fluid than the micromixers known in the art.

### SUMMARY OF INVENTION

Accordingly, the present invention provides a microfluidic or nanofluidic apparatus which comprises:
a) a reservoir for one or more fluids;
b) a mixing device configured to mix the one or more fluids within the reservoir; and
c) a microfluidic or nanofluidic device in fluid connection with the reservoir;
   wherein the reservoir is suitable for containing a total volume of fluid of from 100 microliters to 100 milliliters.

This apparatus enables significantly larger volumes of fluid to be collected and mixed within a microfluidic device than in known microfluidic devices which comprise micromixers which can only contain and mix nanoliters to a few microliters of fluid. A further advantage is that the larger scale of the mixing device of the present invention means that this mixing device is much more straightforward to manufacture and is more easily integrated with microfluidic devices than the known microscale mixers.

The term reservoir is not especially limiting and covers any body of fluid. The fluid may be a liquid or a gas but is typically a liquid. The fluid may be any fluid to be used with a microfluidic or nanofluidic device. Examples include samples comprising one or more proteins, polypeptides, peptides, oligonucleotides, reagents, buffers, wash solutions, or small molecules. The fluid may be a suspension of solid particles. The fluid may be a suspension of beads, preferably magnetic beads. Alternatively, the fluid may comprise a bodily fluid such as blood, plasma, urine or cerebrospinal fluid or may be a suspension of cells or cell components.

The reservoir is usually a container for holding the one or more fluids, which typically comprises a base and one or more side walls extending from the base to form an opening.

In a preferred embodiment the container comprises a tubular portion having a first end and a second end, wherein the first end is attached to a surface of the microfluidic or nanofluidic device such that the surface of the microfluidic or nanofluidic device forms a base for the container and the tubular portion provides the side walls of the container, and wherein the second end provides the opening of the container.

The container may further comprise a closure for closing the opening of the container. The closure is not especially limiting provided that it can close the opening of the container. The closure may be removable. For example, the second end of the tubular portion of the container may comprise a thread portion and the closure may be screwed onto the thread portion. Alternatively, the closure may be attached to the opening of the container, in which case the closure is moveable from a closed position to an open position. For example the closure may be attached to the opening of the container by means of a hinge. The closure may be any kind of cap, lid, cover, or plug.

The mixing device is not especially limited and includes any device suitable for mixing one or more fluids within the reservoir. The mixing device may be a mechanical mixing device, a magnetic mixing device or a sonicator. In a preferred embodiment the mixing device is a mechanical mixing device which comprises a rotatable shaft provided with one or more blades. A motor may be provided for rotating the shaft to facilitate mixing.

In a particularly preferred embodiment the motor is provided on an upper surface of the closure and the rotatable shaft is connected to the motor and extends through the closure such that the one or more blades provided on the shaft are situated within the container when the container is closed by the closure.

The container may either be situated directly onto a surface of the microfluidic or the nanofluidic device or may be situated within a depression on a surface of the microfluidic or nanofluidic device. Where the container comprises a tubular portion, preferably the first end of the tubular portion is situated within the depression on the surface of the microfluidic device.

Typically, the depression has a depth of from 1 to 5 mm, preferably about 3 mm.

Microfluidic devices are devices for handling liquid which comprise at least one channel having at least one dimension of less than 1 mm. Nanofluidic devices are devices for handling liquid which comprise at least one channel having at least one dimension of less than 1 μm. Otherwise, the architecture of the microfluidic or nanofluidic device is not especially limited.

Preferably the microfluidic or nanofluidic device comprises at least one channel having an internal diameter of from 1 μM to 300 μM.

The microfluidic or nanofluidic apparatuses according to the present invention may also comprise macroscale features. In particular, the microfluidic or nanofluidic apparatus of the invention comprises a reservoir suitable for containing a total volume of fluid of from 100 microliters to 100 milliliters and a mixing device configured to mix the one or more fluids within the reservoir.

The microfluidic or nanofluidic device is in fluid connection with the reservoir. This means that fluid can flow between the reservoir and the microfluidic or nanofluidic device.

In a particular embodiment the reservoir further comprises one or more inlets for the one or more fluids. Preferably, the apparatus further comprises one or more fluid sources and a pump for transferring the one or more fluids from one or more fluid sources through the inlets into the container.

The container, mixing device and microfluidic or nanofluidic device may be manufactured from any suitable materials. In a preferred embodiment the microfluidic or nanofluidic device comprises a cyclic olefin copolymer substrate and optionally a polyethylene layer provided on the substrate.

In a further aspect of the invention, the container for the one or more fluids, the mixing device and the microfluidic or nanofluidic device may be provided as a kit of parts. These three components may each be provided separately or two or more of the components may be provided together in appropriate packaging. In a particularly preferred embodiment the container and the mixing device are provided together and the microfluidic or nanofluidic device is provided separately.

In another aspect of the invention is provided a method of manufacturing a microfluidic or nanofluidic apparatus as defined above comprising:
a) providing a microfluidic or nanofluidic device
b) providing a container comprising a base and one or more side walls extending from the base to form an opening, wherein the container is suitable for containing a total volume of fluid of from 100 microliters to 100 milliliters
c) attaching the container to a surface of the microfluidic or nanofluidic device such that the container is in fluid connection with the microfluidic or nanofluidic device
d) attaching a mixing device to the container.
e)

In a preferred method a depression is manufactured in the surface of the microfluidic or nanofluidic device and the container is attached to the depression in step c). Preferably the base of the container is attached to the depression.

Preferably a container is manufactured which comprises a tubular portion having a first end and a second end, wherein the first end is attached to a surface of the microfluidic or nanofluidic device in step c) such that the surface of the microfluidic or nanofluidic device forms a base for the container and the tubular portion provides the side walls of the container, and wherein the second end provides the opening of the container. In this embodiment the first end of the tubular portion is preferably attached to the depression.

Preferably the microfluidic or nanofluidic device comprises a cyclic olefin copolymer substrate and a polyethylene layer provided on the substrate. Typically, the container is attached to a surface of the microfluidic or nanofluidic device by heating the polyethylene layer.

Also provided is the use of an apparatus as defined above for detecting an analyte in a fluid.

### DETAILED DESCRIPTION OF THE INVENTION

The present invention will now be described further by way of example only with reference to the accompanying figures, in which:

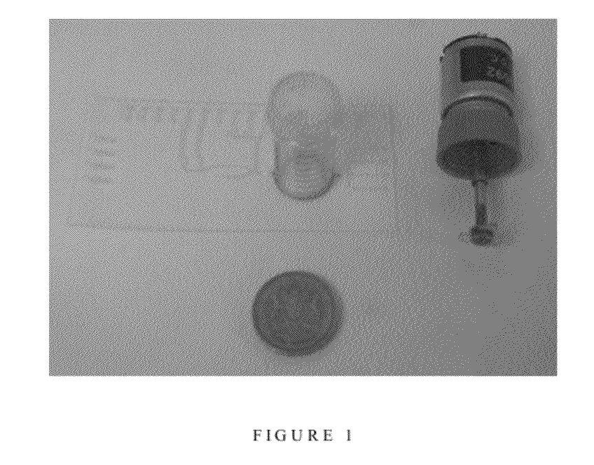

FIG. 1 shows a photograph of an apparatus according to an embodiment of the present invention showing the microfluidic device attached to the container and separately the closure for the container, wherein a motor is attached to the upper surface of the closure and a rotatable shaft is attached to the motor and extends through a cavity in the closure. Blades can be seen attached to the terminus of the shaft. A one pound sterling coin is shown for size comparison.

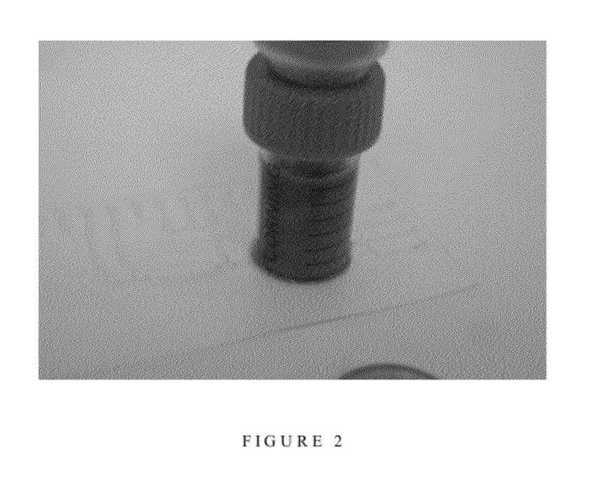

FIG. 2 shows a photograph of an apparatus according to an embodiment of the present invention when in operation. The photograph shows a container containing blue dye and oil. The closure was screwed onto the container such that the mixing device was in the appropriate position to mix the dye and the oil. This photograph was taken within seconds of switching on the motor to activate the mixing device, demonstrating that the mixing device has provided a homogenous mixture of dye and oil.

The term reservoir is not especially limiting and covers any body of one or more fluids. The fluid may be a liquid or a gas but is typically a liquid. The fluid may be any fluid to be used with a microfluidic or nanofluidic device. Examples include samples comprising one or more proteins, polypeptides, peptides, oligonucleotides, reagents, buffers, wash solutions, or small molecules. The fluid may be a suspension of solid particles. The fluid may be a suspension of beads, preferably magnetic beads. Alternatively, the fluid may comprise a bodily fluid such as blood, plasma, urine or cerebrospinal fluid or may be a suspension of cells or cell components.

The fluids to be mixed may be dissimilar fluids, for example oil and a water-based dye.

The reservoir is usually a container for holding the one or more fluids, which typically comprises a base and one or more side walls extending from the base to form an opening. The reservoir may also be a continuous source of fluid, for example a conduit through which fluid flows.

The shape of the container is not especially limited provided the container can hold 100 microliters to 100 milliliters of fluid, more preferably 50 microliters to 50 millileters, more preferably still 1 to 10 millileters, most preferably about 5 millileters.

The container may be a test tube, an Eppendorf™ tube or a custom designed reagent container. The container may be manufactured from any suitable material, typically a polymer.

In a preferred embodiment the container comprises a tubular portion having a first end and a second end, wherein the first end is attached to a surface of the microfluidic or nanofluidic device such that the surface of the microfluidic or nanofluidic device forms a base for the container and the tubular portion provides the side walls of the container, and wherein the second end provides the opening of the container.

The shape of the tubular portion is not particularly limited. Typically the tubular portion has a substantially circular cross-section, but tubular portions having alternative cross-sectional shapes are envisaged. For example, the tubular portion may have a substantially square, rectangular or oval cross section. The tubular portion typically has substantially the same internal diameter along its length but may comprise a narrower portion or a wider portion. In one embodiment the tubular portion is prepared by sectioning an Eppendorf™ tube near the base of the tube.

The container may further comprise a closure for closing the opening of the container. The closure is not especially limited provided that it can close the opening of the container. The closure may be removable. For example, the second end of the tubular portion of the container may comprise a thread portion and the closure may be screwed onto the thread portion. Alternatively, the closure may be attached to the opening of the container, in which case the closure is moveable from a closed position to an open position. For example the closure may be attached to the opening of the container by means of a hinge. The closure may be any kind of cap, lid, cover, or plug.

The mixing device is not especially limited and includes any device suitable for mixing one or more fluids in the reservoir.

The device may mix the components of a single fluid to provide a homogenous fluid. For example the fluid may comprise blood components and magnetic beads and the device may mix this fluid such that the magnetic beads form complexes with the blood components, thus providing a homogeneous fluid comprising magnetic bead: blood component complexes which can then be analysed by the microfluidic device. Another example may be a fluid comprising solid particles, wherein mixing is required to provide a stable suspension of the particles.

Alternatively, two or more different fluids may be mixed by the mixing device within the reservoir. For example, each fluid may comprise a different reactant for a reaction. The fluids may be mixed in the reservoir, leading to reaction of the reactants to form product. The product and/or any remaining reactants may then be analysed in the microfluidic device. In another embodiment one or more of the fluids may contain analytes of interest and one or more other fluids may comprise solvents or other reagents required for analysis of the analyte in the microfluidic device.

Numerous possible mixing devices can be envisaged. The mixing device may stir the one or more fluids or may use other methods of agitating the fluids. The mixing device may be a mechanical mixing device, a magnetic mixing device or a sonicator. In a preferred embodiment the mixing device is a mechanical mixing device which comprises a rotatable shaft provided with one or more blades or paddles. The size and shape of the blades is not particularly limited provided the blades can mix the fluids within the reservoir when the shaft rotates. The blades may be provided at any angle to the axis of the shaft which allows for the blades to carry out their mixing function. In certain embodiments this angle may be adjustable. Typically the one or more blades are provided in a plane at right angles to the longitudinal axis to the shaft. The shaft and blade may be manufactured from any suitable material, for example a polymer or metal.

A motor may be provided for rotating the shaft to facilitate mixing. Preferably the motor is a DC motor.

In a particularly preferred embodiment the motor is provided on an upper surface of the closure and the rotatable shaft is connected to the motor and extends through the closure such that the one or more blades provided on the shaft are situated within the container when the container is closed by the closure. In this embodiment a cavity is manufactured in the closure of a suitable size to enable the rotatable shaft to pass through the closure.

The container may be situated either directly onto a surface of the microfluidic or nanofluidic device or within a depression on a surface of the microfluidic or nanofluidic device. Where the container comprises a tubular portion, preferably the first end of the tubular portion is situated within the depression on a surface of the microfluidic device.

Typically, the depression has a depth of from 1 to 5 mm. Preferably, the depression has a depth of about 3 mm.

The size and shape of the depression is not particularly limited. Preferably, the depression has substantially the same cross-sectional shape as the cross-sectional shape of the container. Typically the depression and the container have substantially circular cross-sections. Preferably the depression has substantially the same diameter as the external diameter of the container. If the container comprises a tubular portion then typically the depression has substantially the same diameter as the first end of the tubular portion.

Microfluidic devices according to the present invention comprise at least one channel having at least one dimension of less than 1 mm. Nanofluidic devices according to the present invention comprise at least one channel having at least one dimension of less than 1 μm. Otherwise, the architecture of the microfluidic or nanofluidic device is not especially limited.

The microfluidic or nanofluidic apparatuses according to the present invention may also comprise macroscale features. In particular, the microfluidic or nanofluidic apparatus of the invention comprises a reservoir suitable for containing a total volume of fluid of from 100 microliters to 100 milliliters and a mixing device configured to mix the one or more fluids within the reservoir.

Preferably the microfluidic or nanofluidic device comprises at least one channel having an internal diameter of from 1 μm to 300 μm. In addition to comprising one or more channels the microfluidic or nanofluidic device may further comprise one or more valves to control the flow of fluid through the channels. The device also typically comprises one or more chambers. One or more chambers may be provided for storing reagents or samples or for carrying out measurements on one or more fluids. One or more chambers may additionally be provided for mixing or reaction of samples or reagents within the microfluidic device in addition to the macro scale mixing device. Typically, at least one channel or chamber is in fluid connection with the reservoir.

The one or more fluids may be transferred to the reservoir manually. Alternatively, the fluids to be mixed are provided in one or more fluid sources. The term fluid source is not especially limiting, and includes any source of fluid. Each fluid source is typically a container for holding the fluid, but may be a continuous source of fluid, for example a fluid flowing through a conduit. In a particular embodiment the reservoir further comprises one or more inlets for the one or more fluids, and the fluid sources are in fluid connection with the one or more inlets. Preferably, the apparatus further comprises a pump for transferring the one or more fluids from one or more fluid sources through the inlets into the container.

The container, mixing device and microfluidic or nanofluidic device may be manufactured from any suitable materials. In a preferred embodiment the microfluidic or nanofluidic device comprises a cyclic olefin copolymer substrate and optionally a polyethylene layer provided on the substrate.

In a further aspect of the invention, the container for the one or more fluids, the mixing device and the microfluidic or nanofluidic device may be provided as a kit of parts. These three components may each be provided separately or two or more of the components may be provided together in appropriate packaging. In a particularly preferred embodiment the container and the mixing device are provided together and the microfluidic or nanofluidic device is provided separately.

In another aspect of the invention is provided a method of manufacturing a microfluidic or nanofluidic apparatus as defined above comprising:
a) providing a microfluidic or nanofluidic device
b) providing a container comprising a base and one or more side walls extending from the base to form an opening, wherein the container is suitable for containing a total volume of fluid of from 100 microliters to 100 milliliters
c) attaching the container to a surface of the microfluidic or nanofluidic device such that the container is in fluid connection with the microfluidic or nanofluidic device
d) attaching a mixing device to the container.
e)

Methods of manufacturing microfluidic/nanofluidic devices are well known in the art. Standard methods for manufacturing suitable containers are also employed, or commercially available containers can be suitably adapted. For example Eppendorf™ tubes may be adapted by cutting off the base of the tube to provide a tubular portion.

Any suitable method for attaching the container to the surface of the microfluidic device may be used. Preferably, the base of the container is attached to the surface of the microfluidic or nanofluidic device.

In one embodiment the method comprises a further step of providing a closure to close the opening of the container.

Preferably the mixing device is a mechanical mixing device. Typically this comprises a rotatable shaft provided with one or more blades. In one embodiment a motor is attached to a top surface of the closure, and the rotatable shaft is connected to the motor, wherein the shaft extends through a cavity in the closure such that the one or more blades provided on the shaft are situated within the container. The method of attaching the motor to a top surface of the closure is also not especially limited. For example, the motor may be bolted, nailed or screwed to the surface of the closure.

In a preferred method a depression is manufactured in the surface of the microfluidic or nanofluidic device and the container is attached to the depression in step c).

Preferably a container is manufactured which comprises a tubular portion having a first end and a second end, wherein the first end is attached to a surface of the microfluidic or nanofluidic device in step c) such that the surface of the microfluidic or nanofluidic device forms a base for the container and the tubular portion provides the side walls of the container, and wherein the second end provides the opening of the container. Preferably, in this embodiment the first end of the tubular portion is attached to the depression.

In a preferred embodiment, the depression is manufactured with a covering surface, and the surface of the depression is removed before the tubular portion is attached to the depression. Typically, the top of the depression is sliced off using a scalpel before the tubular portion is pushed into the depression.

Preferably the microfluidic or nanofluidic device comprises a cyclic olefin copolymer substrate and a polyethylene layer provided on the substrate. Typically the container is attached to the depression by applying heat to the polyethylene layer. In a preferred embodiment a soldering iron is used to melt the polyethylene layer and on hardening the polyethylene provides a seal.

Also provided is the use of an apparatus as defined above for detecting an analyte in a fluid. The apparatus may be used to detect low quantities of an analyte in a fluid, and in this case the volume of the fluid in the reservoir is such that sufficient analyte molecules are provided to enable detection of the analyte.

The present invention will now be described further by way of example only.

### EXAMPLES

### Example 1

### Fabrication of a Microfluidic Apparatus Comprising a Macroscale Mixer

The apparatus according to the present invention was designed and fabricated using manual assembly methods. The apparatus comprised an Eppendorf™ tube, sectioned close to the threaded end. A basic DC motor was sourced and bolted to the threaded top. A shaft and blade was fabricated in order to create a mixer.

In order to integrate the blender, a specific mould was created for the microfluidic device. This included a 3 mm deep depression of the same diameter of the OD of the blender (plus 400 microns to take into account the thickness of the cyclic olefin copolymer (COC) material). The manufacture of the chip was the same as current Lab901 technology. When a fluidic subassembly was created, the top of the 3 mm depression was sliced off using a sharp scalpel and the blender pushed into the space. A soldering iron was then used in order to seal around the blender, using the poly ethylene already present on the inner surface of the COC sheet. The apparatus is shown in FIGS. 1 and 2.

1. A microfluidic or nanofluidic apparatus which comprises:
(a) a reservoir for one or more fluids;
(b) a mixing device configured to mix the one or more fluids within the reservoir; and
(c) a microfluidic or nanofluidic device in fluid connection with the reservoir;
wherein the reservoir is suitable for containing a total volume of fluid of from 100 microliters to 100 milliliters.

2. An apparatus according to claim 1 wherein the microfluidic or nanofluidic device comprises at least one channel having an internal diameter of from 1-300 μm.

3. An apparatus according to claim 1, wherein the reservoir is a container for holding the one or more fluids.

4. An apparatus according to claim 3 wherein the container comprises a base and one or more side walls extending from the base to form an opening.

5. An apparatus according to claim 4, wherein the container comprises a tubular portion having a first end and a second end, wherein the first end is attached to a surface of the microfluidic or nanofluidic device such that the surface of the microfluidic or nanofluidic device forms a base for the container and the tubular portion provides the side walls of the container, and wherein the second end provides the opening of the container.

6. An apparatus according to claim 5, wherein the container further comprises a closure for closing the opening of the container.

7. An apparatus according to claim 6 wherein the closure is removable.

8. An apparatus according to claim 6 wherein the closure is attached to the opening of the container but is moveable from a closed position to an open position.

9. An apparatus according to claim 1 wherein the mixing device is a mechanical mixing device, a magnetic mixing device or a sonicator.

10. An apparatus according to claim 9 wherein the mixing device is a mechanical mixing device and wherein the mixing device comprises a rotatable shaft provided with one or more blades.

11. An apparatus according to claim 10 wherein the mixing device further comprises a motor for rotating the shaft to facilitate mixing.

12. An apparatus according to claim 11 wherein the motor is provided on an upper surface of the closure and the rotatable shaft is connected to the motor and extends through the closure such that the one or more blades provided on the shaft are situated within the container when the container is closed by the closure.

13. An apparatus according to claim 12, wherein the container is situated either directly onto a surface of the microfluidic or nanofluidic device or is situated within a depression on a surface of the microfluidic or nanofluidic device.

14. An apparatus according to claim 13 wherein the depression has a depth of from 1 to 5 mm.

15. An apparatus according to claim 13, wherein the depression has substantially the same diameter as the external diameter of the first end of the tubular portion.

16. An apparatus according to claim 1 wherein the reservoir further comprises one or more inlets for the one or more fluids.

17. An apparatus according to claim 16 which further comprises one or more fluid sources and a pump for transferring the one or more fluids from the one or more fluid sources through the inlets into the reservoir.

18. A kit of parts comprising:
(a) a container for one or more fluids as defined in claim 1;
(b) a mixing device; and
(c) a microfluidic or nanofluidic device;
wherein the mixing device is capable of being attached to the container and wherein the microfluidic or nanofluidic device and the container are capable of being connected such that they are in fluid connection.

19. A method of manufacturing a microfluidic or nanofluidic apparatus comprising:
(a) providing a microfluidic or nanofluidic device;
(b) providing a container comprising a base and one or more side walls extending from the base to form an opening, wherein the container is suitable for containing a total volume of fluid of from 100 microliters to 100 mililiters;
(c) attaching the container to a surface of the microfluidic or nanofluidic device such that the container is in fluid connection with the microfluidic or nanofluidic device;
(d) attaching a mixing device to the container.

20. A method according to claim 19 wherein the microfluidic or nanofluidic device comprises a cyclic olefin copolymer substrate and a polyethylene layer provided on the substrate.

21. A method according to claim 20, wherein the container is attached to a surface of the microfluidic or nanofluidic device by applying heat to the polyethylene layer.

22. Use of an apparatus as defined in any of claim 1 for detecting an analyte in a fluid.
